(19) United States
(12) Patent Application Publication
(10) Pub. No.: US 2010/0108989 A1
(43) Pub. Date: May 6, 2010

(54) ELECTROLUMINESCENT POLYMERS AND USE THEREOF

(75) Inventors: Arne Büsing, Frankfurt (DE); Philipp Stössel, Frankfurt (DE)

Correspondence Address: CONNOLLY BOVE LODGE & HUTZ, LLP, P O BOX 2207, WILMINGTON, DE, 19899, US

(73) Assignee: Merck Patent GmbH, Darmstadt (DE)

(21) Appl. No.: 11/721,913

(22) PCT Filed: Dec. 16, 2005
(86) PCT No.: PCT/EP2005/013610

§ 371 Date: Jan. 4, 2010

(30) Foreign Application Priority Data
Dec. 18, 2004 (EP) 04030093.1

(51) Int. Cl. H01L 51/30; C08G 61/10; C07C 13/47; C07C 25/22; C07F 5/02; C07C 69/76; C09K 11/06

(52) U.S. Cl. 257/40; 528/396; 528/397; 528/8; 528/86; 585/26; 570/183; 568/3; 560/80; 252/301.35; 257/E51.027

(57) ABSTRACT

The present invention relates to polymers which contain novel structural units of the formula (1). The inventive materials exhibit better solubility and improved efficiency when used in a polymeric organic light-emitting diode.

For a number of years, broadly based research has proceeded into the commercialization of display and illumination elements based on polymeric (organic) light-emitting diodes (PLEDs). This development was triggered by the fundamental developments which are disclosed in WO 90/13148. Recently, first products (small displays in a shaver and a mobile telephone from PHILIPS N.V.) have even become available on the market. However, distinct improvements in the materials used are still necessary for these displays to provide real competition to the currently market-leading liquid crystal displays (LCDs).

Various material classes have been developed as polymers for full-colour display elements. Examples of useful material classes include polyfluorene, polyspirobifluorene, polydihydrophenanthrene, polyindenofluorene and polyphenanthrene derivatives, or else polymers which contain a combination of these structural elements. In general, polymers which contain poly-para-phenylene (PPP) as a structural element are possible for such a use.

Some of the polymers according to the prior art already exhibit good properties. However, in spite of the advances already achieved, they do not yet meet the demands which are placed on them for high-value PLED applications. In particular, the efficiency of the emitting polymers is still not sufficient for many applications. The combination of polymers with triplet emitters has also to date not yet led to the expected high efficiencies, especially for green emission. A further problem of the polymers according to the prior art is their frequently low solubility. Thus, it is possible to obtain soluble polymers with the abovementioned polymer classes only when they are solubilized by alkyl and/or alkoxy chains. In spite of these substitutions, some of these units, especially dihydrophenanthrene and phenanthrene units, only have a low solubility in the solvents typically used. Therefore, they can be processed either only from ecologically controversial solvents which cannot be used in industrial processing, steps, such as chlorobenzene, or there are no suitable solvents at all, so that remedy is needed here. Substitution by long alkyl and/or alkoxy chains is also undesirable, since they do not realize any electronic function but possibly inhibit charge transport between the polymer chains and reduce the concentration of the functional units in the polymer. Moreover, long alkyl and alkoxy chains function as plasticizers which lower the glass transition temperature of the polymers, which is undesired for the application in organic electronic devices.

Without wishing to be bound to a particular theory, we suspect that the electronic properties of the polymers conjugated throughout according to the prior art are not yet optimal for balanced charge transport. As a result, the electron flow or the hole flow in the device is possibly too great, and a balanced charge equilibrium cannot be established. It may also be sensible for some applications to fully or partly separate the functions of the different units in the polymer, i.e., for example, charge transport and emission, from one another. This is possible in fully conjugated polymers only by, for example, the charge transport units being incorporated into the side chain of the polymer, as described in EP 1263834. However, these functions in the side chain do not have any conjugated sections apart from the functional unit itself, which is again not always sufficient for good charge transport.

It has now been found that, surprisingly, a novel class of polymers in which the conjugation is reduced by the use of certain novel monomer units but is not entirely interrupted has very good properties exceeding the abovementioned prior art. These polymers and their use in PLEDs therefore form part of the subject-matter of the present invention. The novel structural units are suitable in particular as a polymer backbone, particularly in conjunction with another polymer backbone which has distinctly better solubility through the use of the novel units, or else in conjunction with triplet emitters.

The use of certain binaphthyl units in polymers has been described in the literature: thus, X. Wu et al. (Synth. Metals 2001, 121, 1699-1700) describe an alternating copolymer composed of 6,6′-bonded 2,2′-substituted 1,1′-binaphthyl units and substituted phenylene units. The good solubility of the polymer is attributed to the presence of four n-hexyloxy side chains, which indicates that corresponding unsubstituted units do not lead to soluble polymers. Electroluminescence results are not presented. L. Zheng et al. (Chem. Mater. 2000, 12, 13-15) describe a poly(binaphthylvinylene-phenylenevinylene) in which the 1,1′-binaphthyl units are again bonded in the polymer via the 6,6′-position. Here too, alkoxy chains are again needed for the solubility of the polymer. The polymer exhibits light blue fluorescence in solution but bathochromically shifted green-blue emission in a film, so that it is unsuitable for blue emission. Moreover, the external quantum efficiency is very low at 0.1% and the use voltage very high at 6 V. Further similar polymers are known in the literature (for example K. Y. Musick et al., Macromolecules 1998, 31, 2933; Q.-S. Hu et al., Macromolecules 1996, 29, 1082; Q. S. Hu et al., Macromolecules 1996, 29, 5075; L. Ma et al., Macromolecules 1996, 29, 5083; A. K.-Y. Jen, Appl. Phys. Lett. 1999, 75, 3745). 6,6′-Bonded, 1,1′-binaphthyl units appear generally to have the weakness that the solubility of these polymers is insufficient and good solubility is achieved only by the introduction of long alkyl or alkoxy chains. As already mentioned above, these do not contribute anything to the electronic function of the polymer and can even have a disruptive effect on the charge transport between the polymer chains and the glass transition temperature.

U. Anton and K. Müllen (Macromolecules 1993, 26, 1248) describe polynaphthalenes in which the monomers used are unsubstituted 4,4′-binaphthyl and alkyl-substituted 1,5-dibromonaphthalene. Even in the presence of hexyl chains as substituents on the dibromonaphthalene, the solubility is still so low that only oligomers but no polymers are obtained. Even with two dodecyl chains, only low molecular weights (Mₙ=5900 g/mol) are obtained. The use of such oligomers in organic electronic devices is not described.

DE 4024647 describes 4,4′-bonded oligo- and polynaphthalenes which are suitable as materials for thermal insulation and as electrode materials. A preference for substitution in the 2,2′-position is not evident. The use of these polymers in organic light-emitting diodes or as semiconductor materials in other organic electronic devices is not described.

P. V. Bedworth and J. M. Tour (Macromolecules 1994, 27, 622-624) describe helical oligomers based on enantiomerically pure substituted 4,4′-bonded 2,2′-dimethoxy-1,1′-binaphthylene (M_{w}=14700 g/mol). Suitability for organic electronic devices is not evident, nor would be expected by virtue of the dimethoxy substitution, since the methoxy groups are firstly suspected to lower the thermal stability of the polymers and secondly shift the emission colour bathochromically, so that these polymers will be unsuitable for many applications. However, the presence of the methoxy groups appears to be indispensable for the synthetic obtainability of these polymers.

V. Percec et al. (Polymer Bulletin 1992, 29, 271-276) describe an alternating polymer composed of unsubstituted 4,4′-(1,1′-binaphthyl) and 4,4′-(3,3′-diphenyl)biphenyl. However, only degrees of polymerization of less than 10 are achieved, so that the resulting structures should rather be referred to as oligomers. Use in organic electronic devices is not described, but it is not to be suspected that oligomers with such a low molecular weight are suitable therefor.

X. Zhan et al. (Chem. Mater. 2003, 15, 1963-1969) describe blue-emitting polymers based on cyanostilbenes which are joined by 3,3′-(1,1′-binaphthyl). Solubility is achieved by introducing hexoxy chains in the 2,2′-position of the binaphthyl. However, these polymers exhibit only poor results in organic electronic devices. Thus, for the best devices, only low brightnesses are achieved at a voltage of 18 V, and a quantum efficiency of only 0.2%. These polymers are therefore unsuitable for industrial application.

The substitution of the binaphthyl units in the 2- or 2,2′-position with alkyl or aryl groups and the bonding in the polymer in the 4,4′-position has surprisingly been found to be particularly suitable in comparison to the use of unsubstituted or differently substituted binaphthyl units and for bonding via other positions. This allows soluble polymers with better optical and electronic properties to be synthesized particularly efficiently. Therefore, polymers which contain such units form part of the subject-matter of the present invention.

For clarity, the positions of 1,1′-binaphthyl are illustrated in the scheme which follows:

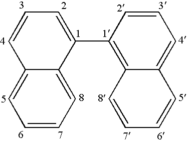

The invention provides polymers containing at least 1 mol %, preferably at least 5 mol %, more preferably at least 10 mol %, of a first repeat unit of the formula (1)

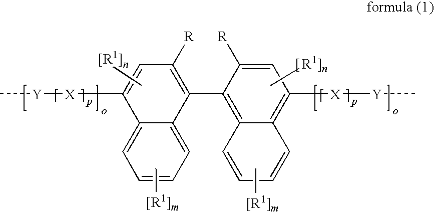

where the symbols and indices used are each defined as follows:
R is the same or different at each instance and is H, a straight alkyl chain having 1 to 40 carbon atoms or a branched or cyclic alkyl chain having 3 to 40 carbon atoms, each of which may be substituted by R¹ and in which one or more nonadjacent carbon atoms may also be replaced by N—R¹, O, S, O—CO—O, CO—O, —CR¹═CR¹— or —C≡C—, with the proviso that the heteroatoms are not bonded directly to the naphthyl unit, and in which one or more hydrogen atoms may also be replaced by F, Cl, Br, I or CN, or an aromatic or heteroaromatic ring system which has from 5 to 40 aromatic ring atoms and may also be substituted by one or more R¹ radicals; the two R radicals together may also form a further ring system; with the proviso that at least one of the two R radicals is not H;
X is the same or different at each instance and is —CR¹═CR¹—, —C≡C— or N—Ar;
Y is the same or different at each instance and is a bivalent aromatic or heteroaromatic ring system which has 5 to 40 aromatic ring atoms and may be substituted by one or more R¹ radicals or unsubstituted;
R¹ is the same or different at each instance and is H, F, Cl, Br, I, CN, N(R²)₂, Si(R²)₃, B(R²)₂, a straight alkyl or alkoxy chain having 1 to 40 carbon atoms or a branched or cyclic alkyl or alkoxy chain having 3 to 40 carbon atoms, in which one or more nonadjacent carbon atoms may also be replaced by N—R², O, S, O—CO—O, CO—O, —CR¹═CR¹— or —C≡C— and in which one or more hydrogen atoms may also be replaced by F, Cl, Br, I or CN, or an aryl, aryloxy or heteroaryl group which has 5 to 40 aromatic ring atoms and may also be substituted by one or more nonaromatic R¹ radicals; two or more of the R¹ radicals together may also form a ring system;
R² is the same or different at each instance and is H or an aliphatic or aromatic hydrocarbon radical having 1 to 20 carbon atoms;
Ar is the same or different at each instance and is a monovalent aromatic or heteroaromatic ring system which has 5 to 40 aromatic ring atoms and may be substituted by R¹ or unsubstituted;
n is the same or different at each instance and is 0 or 1;
m is the same or different at each instance and is 0, 1, 2, 3 or 4;
o is the same or different at each instance and is 0, 1 or 2;
p is the same or different at each instance and is 0 or 1;
   in formula (1) as well as in all further formulae, the dashed bond is the linkage in the polymer;
   and also containing at least 1 mol % of a second repeat unit which is either the same as a repeat unit of the formula (1) or is different.

In the context of the present invention, a C₁- to C₄₀-alkyl group in which individual hydrogen atoms or CH₂ groups may also be substituted by the above-mentioned groups is more preferably understood to mean the methyl, ethyl, n-propyl, i-propyl, n-butyl, i-butyl, s-butyl, t-butyl, 2-methylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals. A C₁- to C₄₀-alkoxy group is more preferably understood to mean methoxy, ethoxy, n-propoxy, i-propoxy, n-butoxy, i-butoxy, s-butoxy, t-butoxy or 2-methylbutoxy. An aromatic or heteroaromatic system which has 1-30 carbon atoms and may also be substituted in each case by the abovementioned R¹ radicals and which may be attached via any positions to the aromatic or heteroaromatic is understood in particular to mean groups which are derived from benzene, naphthalene, anthracene, phenanthrene, pyrene, chrysene, perylene, fluoranthene, naphthacene, pentacene, benzpyrene, biphenyl, biphenylene, terphenyl, terphenylene, fluorene, spirobifluorene, dihydrophenanthrene, dihydropyrene, tetrahydropyrene, cis- or trans-indenofluorene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazineimidazole, quinoxalineimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

In the context of this invention, an aromatic or heteroaromatic ring system should be understood to mean a system which does not necessarily contain only aromatic or heteroaromatic groups, but in which a plurality of aromatic or heteroaromatic groups may also be interrupted by a short nonaromatic unit (<10% of the atoms other than H, preferably <5% of the atoms other than H), for example sp³-hybridized C, N, etc. For example, systems such as 9,9′-spirobifluorene, 9,9-diarylfluorene, triarylamine, etc. should thus also be understood to be aromatic ring systems. The aromatic groups contain at least 6 carbon atoms and the heteroaromatic groups at least 2 carbon atoms and at least one heteroatom, the sum of carbon atoms and heteroatoms adding up to at least 5.

Units of the formula (1) are present in two enantiomeric forms. It is equally in accordance with the invention to use the racemate, i.e. the 1:1 mixture of the two enantiomers, or one of the two enantiomers in enriched or isolated form. The sequence of the stereocentres in polymers is referred to as tacticity.

One aspect of the invention relates to conjugated polymers. Another aspect of the invention relates to part-conjugated polymers. Yet a further aspect of the invention relates to nonconjugated polymers. Preference is given to conjugated or part-conjugated polymers.

Conjugated polymers in the context of this invention are polymers which contain, in the main chain, mainly sp²-hybridized carbon atoms which may also be replaced by corresponding heteroatoms. In the simplest case, this means alternating presence of double (or else triple) and single bonds in the main chain. “Mainly” means that defects which occur naturally and lead to interruptions in conjugation do not invalidate the term “conjugated polymer”. Furthermore, this application text likewise refers to polymers as conjugated when, for example, arylamine units and/or conjugated heterocycles (i.e. conjugation via N, O or S atoms) and/or organometallic complexes (i.e. conjugation via the metal atom) are present in the main chain. In contrast, units such as simple alkyl bridges, (thio)ether, ester, amide or imide linkages, for example, would be defined unambiguously as nonconjugated segments. A part-conjugated polymer shall be understood to mean a polymer in which prolonged conjugated sections in the main chain are interrupted by nonconjugated sections, or which contains prolonged conjugated sections in the side chains of a polymer nonconjugated in the main chain.

However, it should be pointed out here that units of the formula (1) reduce the conjugation of a polymer chain otherwise conjugated throughout, since the two naphthyl units form an angle with one another in the order of magnitude of 60 to 120°. However, the conjugation is not entirely interrupted by such units.

Preference is given to inventive polymers in which, for units of the formula (1):
R is the same or different at each instance and is H, a straight alkyl chain having 1 to 10 carbon atoms or a branched or cyclic alkyl chain having 3 to 10 carbon atoms, in which one or more nonadjacent carbon atoms may also be replaced by —CH═CH— or —C≡C— and in which one or more hydrogen atoms may also be replaced by F or CN, or an aromatic or heteroaromatic ring system which has 5 to 20 aromatic ring atoms and may also be substituted by one or more nonaromatic R¹ radicals; the two R radicals together may also form a further ring system, with the proviso that at least one R radical is not H;
Y is the same or different at each instance and is a bivalent aryl or heteroaryl group which has 5 to 25 aromatic ring atoms and may be substituted by one or more R¹ radicals;
Ar is the same or different at each instance and is a monovalent aryl or heteroaryl group which has 5 to 20 aromatic ring atoms and may be substituted by R¹ or unsubstituted;
R¹ is the same or different at each instance and is H, F, N(R²)₂, a straight alkyl chain having 1 to 10 carbon atoms or a branched alkyl chain having 3 to 10 carbon atoms, in which one or more nonadjacent carbon atoms may also be replaced by —CR¹═CR¹— or —C≡C— and in which one or more hydrogen atoms may also be replaced by F, or an aryl or heteroaryl group which has 5 to 20 aromatic ring atoms and may also be substituted by one or more nonaromatic R¹ radicals; two or more of the R¹ radicals together may also form a ring system;
n at each instance is 0;
m is the same or different at each instance and is 0, 1 or 2;
o is the same or different at each instance and is 0 or 1;
   the further symbols and indices are as defined above under formula (1).

Particular preference is given to inventive polymers in which, for units of the formula (1):
R is the same or different at each instance and is H or an aromatic or heteroaromatic group which has 5 to 15 aromatic ring atoms and may also be substituted by one or more nonaromatic R¹ radicals; the two R radicals together may also form a further ring system; with the proviso that at least one of the R radicals is not H;
X is the same or different at each instance and is —CH═CH—, —C≡C— or N—Ar;
Y is the same or different at each instance and is a bivalent aryl or heteroaryl group which has 6 to 15 carbon atoms and may be substituted by one or more nonaromatic R¹ radicals;
Ar is the same or different at each instance and is a monovalent aryl or heteroaryl group which has 5 to 15 aromatic ring atoms and may be substituted by nonaromatic R¹ radicals;
n at each instance is 0;
m at each instance is 0,
o is the same or different at each instance and is 0 or 1;
   the further symbols and indices are each as defined above.

The reason for the preference for aromatic R radicals is the better electronic properties and the higher thermal stability of the polymers.

In a preferred embodiment of the invention, the two R radicals are the same. In a further preferred embodiment of the invention, one of the two R radicals is H and the other an aromatic or heteroaromatic ring system as described above.

It is equally possible that the X and Y substituents are each the same or else different or only occur on one side.

In addition to units of the formula (1), the inventive polymers preferably also contain further structural elements which are different from units of the formula (1). The further structural elements are preferably conjugated. Reference is made here in particular also to the relatively comprehensive lists in WO 02/077060, in WO 05/014689 and the references cited therein. The further structural units are preferably selected from the classes described below:

Group 1: Aromatic Units which Typically Constitute the Polymer Backbone:

The polymer backbone serves generally as a “matrix” for the functional units in the polymer, for example charge transport or emission units. Units of this group are aromatic, carbocyclic structures which have 6 to 40 carbon atoms and may be substituted or unsubstituted, possible substituents being the abovementioned R¹ radicals. Fluorene derivatives (for example EP 0842208, WO 99/54385, WO 00/22027, WO 00/22026, WO 00/46321) are useful here. In addition, spirobifluorene derivatives (for example EP 0707020, EP 0894107, WO 03/020790) are also a possibility. Polymers which contain a combination of these two monomer units have also already been proposed (WO 02/077060). WO 05/014689 describes dihydrophenanthrene derivatives. Also useful are cis- or trans-indenofluorene derivatives (for example WO 04/041901, WO 04/113412), but also 1,4-phenylene derivatives, 4,4′-biphenylylene derivatives, 4,4″-terphenylylene derivatives, 2,7-phenanthrene derivatives (for example DE 0102004020298.2), dihydropyrene or tetrahydropyrene derivatives and further aromatic structures which are not detailed explicitly. Units from group 1 are thus preferably selected from the group of the fluorene derivatives, the spirobifluorene derivatives, the dihydrophenanthrene derivatives, the cis- or trans-indenofluorene derivatives, the 1,4-phenylene derivatives, the 4,4′-biphenylylene derivatives, the 4,4″-terphenylylene derivatives, the 2,7-phenanthrene derivatives, the dihydropyrene or tetrahydropyrene derivatives. Particularly preferred units from this group are selected from spirobifluorene, fluorene, dihydrophenanthrene, cis-indenofluorene, trans-indenofluorene and 2,7-phenanthrene, which may be substituted by R¹ or unsubstituted.

Group 2: Units which Change the Morphology or the Emission Colour:

These units are preferably selected from the group of the R¹-substituted or unsubstituted fused aromatic structures having 6 to 40 carbon atoms or tolane, stilbene or bisstyrylarylene derivatives, in particular 1,4-naphthylene, 1,4- or 9,10-anthrylene, 1,6- or 2,7- or 4,9-pyrenylene, 3,9- or 3,10-perylenylene, 4,4′-tolanylene, 4,4′-stilbenzylene or 4,4″-bisstyrylarylene derivatives.

Group 3: Units which Increase the Hole Injection and/or Transport Properties of the Polymers:

These are generally aromatic amines or phosphines or electron-rich heterocycles. They are preferably selected from the group of the R¹-substituted or unsubstituted triarylamines, benzidines, N,N,N′,N′-tetraaryl-para-phenylenediamines, triarylphosphines, phenothiazines, phenoxazines, dihydrophenazines, thianthrenes, dibenzo-p-dioxins, phenoxathiines, carbazoles, azulenes, thiophenes, pyrroles, furans and further O-, S- or N-containing heterocycles with high-lying HOMO (HOMO=highest occupied molecular orbital). These units may be incorporated into the main chain or into the side chain of the polymer. Depending on the structure, the polymer backbone is even capable of conducting holes sufficiently well that units from group 3 do not necessarily have to be present.

Group 4: Units which Increase the Electron Injection and/or Transport Properties of the Polymers:

These are generally electron-poor aromatics or heterocycles. They are preferably selected from the group of the R¹-substituted or unsubstituted pyridines, pyrimidines, pyridazines, pyrazines, triazines, oxadiazoles, quinolines, quinoxalines or phenazines, but compounds such as triarylboranes and further O-, S- or N-containing heterocycles with low-lying LUMO (LUMO=lowest unoccupied molecular orbital) are also useful. Depending on the structure, the polymer backbone itself is even capable of conducting electrons sufficiently well that units from group 4 do not necessarily have to be present.

Group 5: Units which have Combinations of Individual Units of Group 3 and Group 4:

It may be preferred when units are present in the inventive polymers in which structures which have hole transport properties and structures which have electron transport properties are bonded directly to one another, i.e. structures from the abovementioned groups 3 and 4. Many of these units shift the emission colour into the green, yellow or red; their use is thus suitable for obtaining different emission colours from originally blue-emitting polymers.

Group 6: Units which Emit Light from the Triplet State:

Structural units from this group can emit light from the triplet state with high efficiency even at room temperature and exhibit electrophosphorescence instead of electrofluorescence. Suitable for this purpose are firstly compounds which contain heavy atoms having an atomic number of more than 36. Particularly suitable compounds contain d or f transition metals which fulfil this condition. Very particular preference is given to structural units which contain elements of group 8 to 10 (Ru, Os, Rh, Ir, Pd, Pt), in particular Ir or Pt. These metal complexes may be bonded into the main chain and/or into the side chain of the polymer. It has also been found to be suitable to incorporate such metal complexes at branching points in the polymer, as described, for example, in DE 102004032527.8. When units from group 6 are present, it may be preferred simultaneously also to use units from group 7. However, even without such units from group 7, it is possible to achieve very high efficiencies with triplet emitters.

Group 7: Units which Promote the Transfer from the Singlet to the Triplet State:

For the use of triplet emitters, it may be preferred to use further structural elements in a promoting role, which improve the transition from the singlet to the triplet state and thus the electrophosphorescence properties. Useful for this purpose are, for example, carbazole units, as described in WO 04/070772 and WO 04/113468, but also keto, phosphine oxide, sulphoxide or sulphone units, as described in the unpublished application DE 10349033.7, or silane units as described in WO 05/040302. The units from group 7 are thus preferably selected from the group of the carbazoles, the bridged carbazole dimers, the ketones, phosphine oxides, sulphoxides, sulphones or the silanes.

Preference is given to polymers which, as well as structural units of the formula (1), additionally contain one or more units selected from groups 1 to 7. It may also be advantageous when more than one structural unit from one of groups 1 to 7 is present at the same time.

Particular preference is given to polymers which, as well as units of the formula (1), also contain units from group 1, most preferably at least 30 mol % of these units.

Preference is also given to polymers which, as well as units of the formula (1), also contain units from group 3 and/or 4, more preferably from group 3, most preferably at least 5 mol % of these units.

The proportion of units of the formula (1) is preferably at least 5 mol %, more preferably at least 10 mol %, most preferably at least 20 mol %. This preference applies in particular when the units of the formula (1) are the polymer backbone. In the case of other functions, other fractions may be preferred. For other applications, for example for organic transistors, the preferred fraction may again be different, for example up to 100 mol %, when the hole-conducting units are of the formula (1).

The inventive polymers have generally from 10 to 10 000, preferably from 50 to 5000, more preferably from 50 to 2000 repeat units.

The solubility of the polymers is ensured partly by the substituents R¹ on the further structural units present. However, as already described above, long alkyl or alkoxy substituents in the polymer are not always desirable and also do not always lead to the desired solubility.

The substituents R and, if present, R¹ on formula (1) also contribute to the solubility of the polymers. However, even the basic structure of the formula (1) itself contributes to considerably better solubility of the polymer, so that the presence of long-chain substituents R¹ on the naphthyl units is not necessary, nor is it preferred. The presence of one or two R groups is sufficient for good solubility and also distinctly increases the solubility of the overall polymer. It is not necessary that R contains long alkyl chains, but rather it is sufficient when either purely aromatic substituents are used here, or aromatic substituents which are substituted with only short alkyl chains, for example tert-butyl, or short-chain alkyl substituents. Polymers in which only one of the R radicals constitutes a group other than hydrogen also have very good solubility.

Depending on the substitution pattern, units of the formula (1) are suitable for various functions in the polymer. These units may be used with preference as the polymer backbone or in combination with a polymer backbone as described above to increase the solubility or else as a hole conductor or as an emitter. The polymer backbone serves generally as a “matrix” for the functional units in the polymer, for example charge transport or emission units. Which compounds are especially suitable for which function is described in particular by the X and Y groups. The substituents R and R¹ have a less marked influence on the function of the units of the formula (1).

For instance, for use as a polymer backbone or in combination with a polymer backbone to increase the solubility, preferably:
p at each instance is 0,
   i.e. it is a purely aromatic structural unit.

For the use of units of the formula (1) as hole-transporting units, preferably:
p is the same or different at each instance and is 0 or 1, where at least one p
o is the same or different at each instance and is 0, 1 or 2, where o does not equal 0 when the corresponding p=1;
X at each instance is N—Ar,
   i.e. they are triarylamine derivatives of binaphthyl.

For the use of units of the formula (1) as emitters, preferably:
p is the same or different at each instance and is 0 or 1, where at least one p=1;
o is the same or different at each instance and is 0, 1 or 2, where o does not equal 0 when the corresponding p=1;
X is the same or different at each instance and is —CR¹═CR¹—, —C≡C— or N—Ar, where at least one X is —CR¹═CR¹— or —C≡C—,
   i.e. they are diarylvinylene or diarylacetylene derivatives of binaphthyl in the widest sense, which may also additionally contain triarylamine units.

Examples of preferred units of the formula (1) are structures according to the Examples (1) to (21) depicted, the linkage within the polymer in each case being via the 4,4′-positions of the binaphthyl units, as indicated by means of the dashed bonds. Possible further substituents R¹ are generally not or not always shown for better clarity. Examples (1) to (9) are examples of backbone units, Examples (10) to (17) are examples of emitting units and Examples (18) to (21) are examples of hole-conducting units.

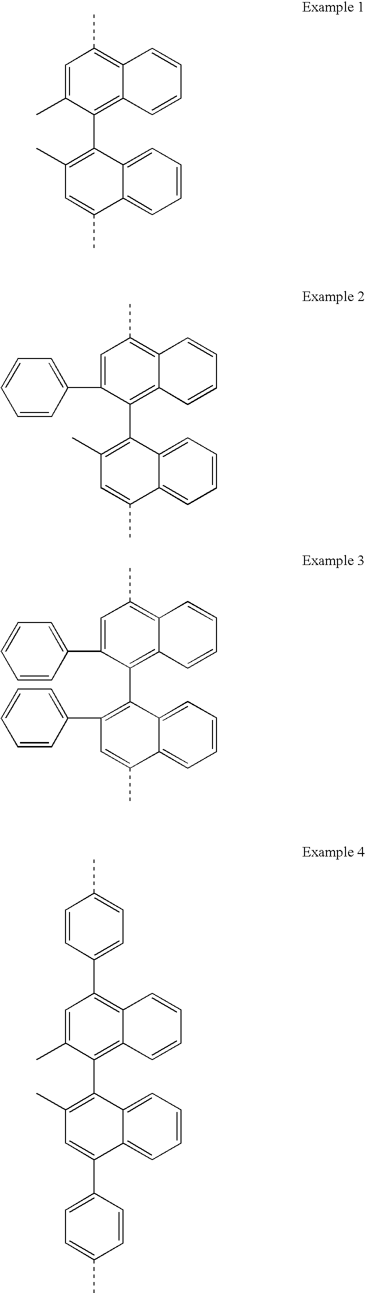

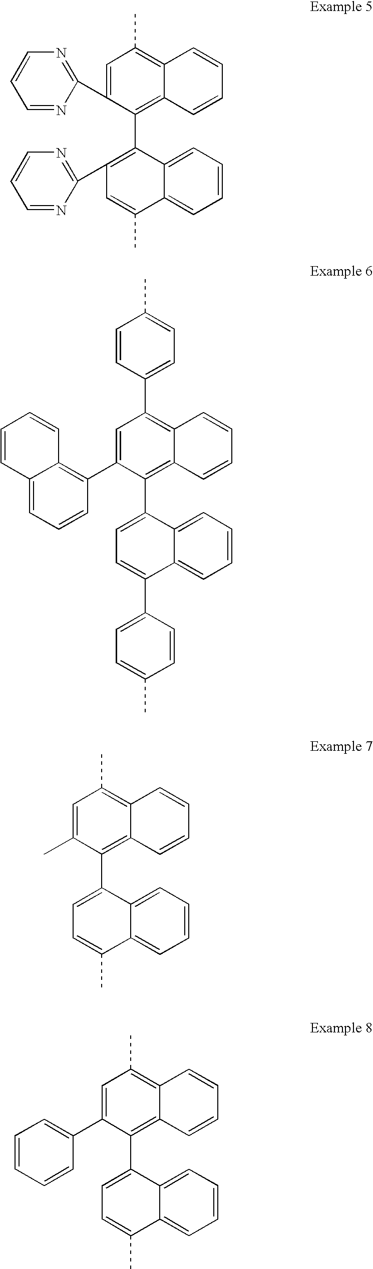

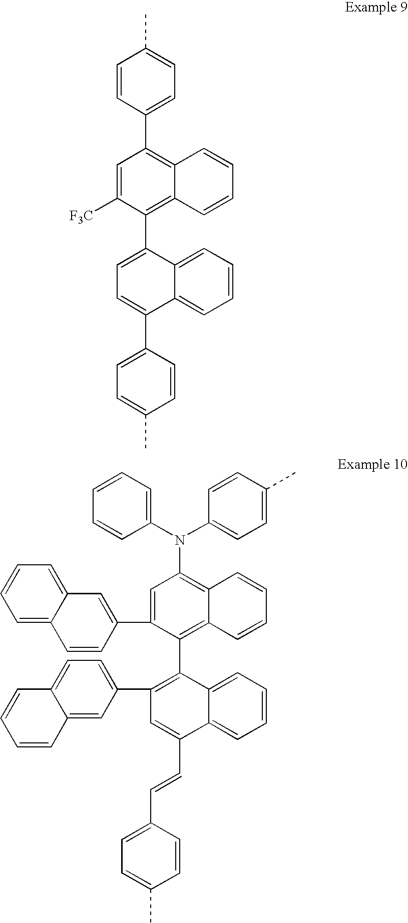

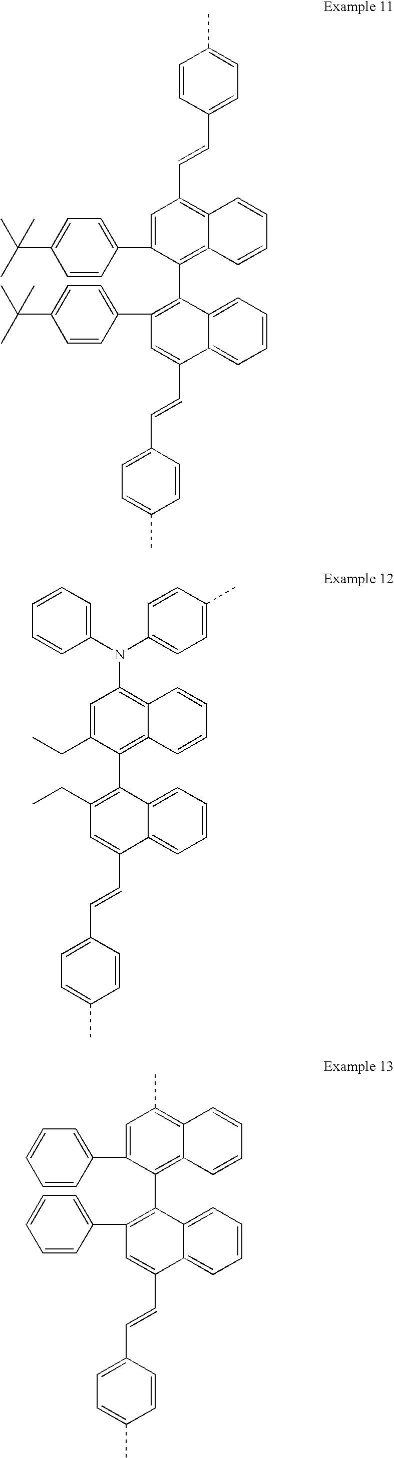

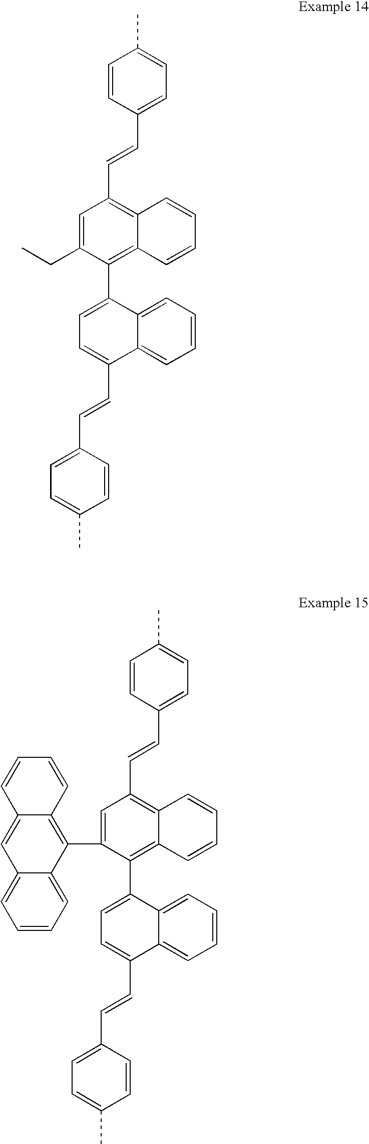

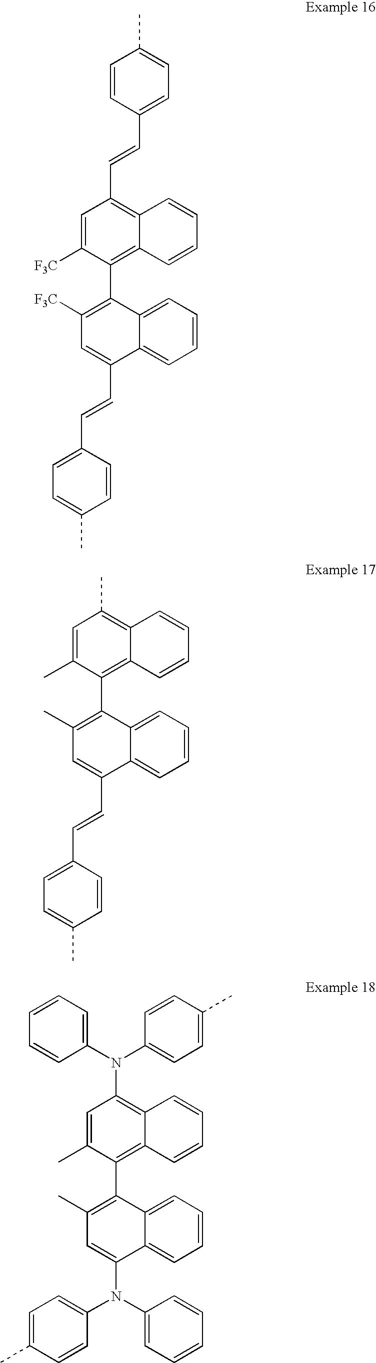

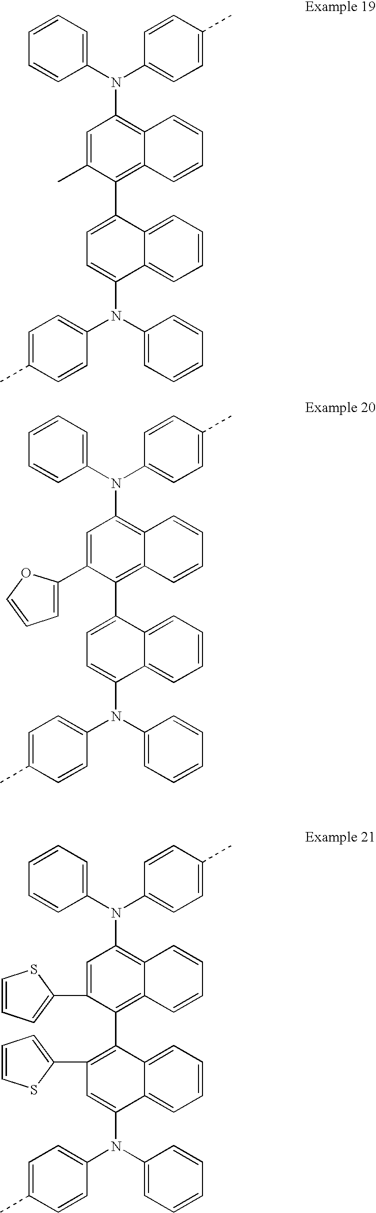

The inventive polymers are homopolymers or copolymers. Inventive copolymers may, as well as one or more structures of the formula (1), have one or more further structures, for example from the abovementioned groups 1 to 7.

The inventive copolymers may have random, alternating or block-like structures or else possess a plurality of these structures in alternation. This can also relate to the tacticity. The way in which copolymers with block-like structures can be obtained is described, for example, in detail in WO 05/014688. The use of different structural elements allows properties such as solubility, solid-phase morphology, colour, charge-injection and -transport properties, electrooptical characteristics, etc., to be adjusted. The polymers may likewise have a linear or branched structure or else contain dendritic structures.

The precise structure of the polymer and the exact arrangement of the repeat units in the polymer may be crucial for the function. Without wishing to be bound to a particular theory, we suspect that, in many fully conjugated polymers, the charge mobility either for holes and/or for electrons is too high, so that a balanced charge equilibrium cannot be established overall. As a consequence, one charge carrier is unnecessarily present in too high a fraction, which might in turn lead to temperature increase as a result of high currents and side reactions in the polymer or in the device, which lowers the lifetime of the device. Poorly balanced charge transport also makes the efficiency lower than it might be in the case of balanced hole and electron transport. Introduction of the inventive units of the formula (1) and the controlled incorporation of the individual functionalities into the polymer can remedy this problem: reduction but not full interruption of the conjugation and thus of charge transport through the units of the formula (1) allows the charge carrier mobility for the corresponding charge carriers to be adjusted in a controlled manner and thus the charge balance in the electronic device to be improved. Such structures can be incorporated in a controlled manner, for example, by Suzuki coupling, in which case the selective reaction of halogens with boronic acid derivatives can precisely control the arrangement of the monomers.

Such a structure, in which the emitting sections of the polymer are limited approximately between two units of the formula (1), also has the advantage that the conjugation length is defined here, which in turn leads to a less broadband emission and thus to greater colour purity.

Especially for polymers which emit light from the triplet state, there appears to be a preference for using polymer which is not fully conjugated. Without wishing to be bound to a particular theory, we suspect that, in conjugated polymers, the energy transfer to the triplet emitter is frequently incomplete, or that transfer from the triplet emitter back to the polymer can take place. This problem can be reduced by using units of the formula (1).

The inventive polymers are generally prepared by polymerizing one or more monomer types, of which at least one monomer in the polymer leads to units of the formula (1). Some polymerization reactions which lead to C—C or to C—N bond formations have been found to be particularly useful here:

(A) polymerization according to SUZUKI;
(B) polymerization according to YAMAMOTO;
(C) polymerization according to STILLE;
(D) polymerization according to HARTWIG-BUCHWALD.

The way in which the polymerization can be carried out by these methods and the polymers can be removed from the reaction medium and purified is described, for example, in WO 03/048225 and WO 04/022626. The way in which particularly pure polymers can be obtained is described in the unpublished application EP 04023475.9.

Monomers which lead to structural units of the formula (1) in inventive polymers are binaphthyl derivatives which are substituted suitably in the 2- and/or 2′-position and, at the 4,4′-position (or in a suitable position on the Y group if present), have suitable functionalities which allow this monomer unit to be incorporated into the polymer. These monomers are obtainable, for example, in the following way: commercial 2,2′-dihydroxy-1,1′-binaphthyl is converted to the corresponding triflate and this is converted in a Grignard cross-coupling reaction to the correspondingly substituted binaphthyls (scheme 1). The products may then be brominated regioselectively at the 4,4′-position and the bromides in turn converted to the boronic acids required for the Suzuki polymerization, for example.

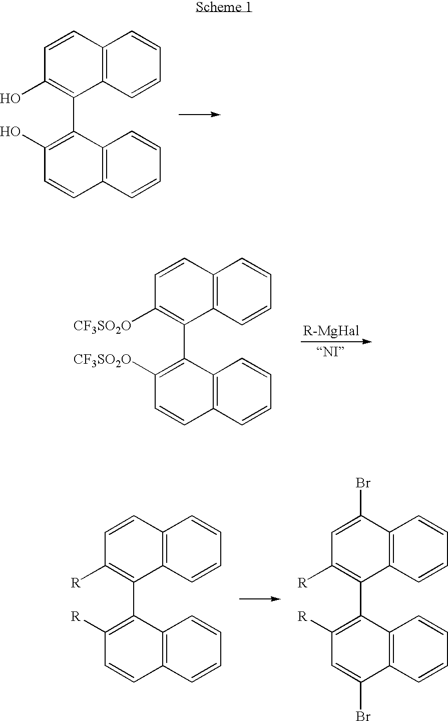

A further route is shown in scheme 2:

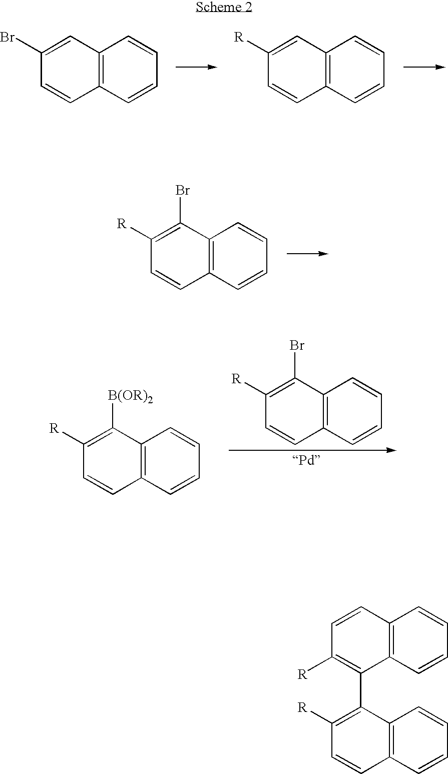

The somewhat greater synthetic complexity in scheme 2 is justified by the lower costs of the raw materials and by the fact that the asymmetric binaphthyls are obtainable by this route.

Monomers which lead to units of the formula (1) in the polymer are novel.

The invention further provides for the use of bifunctional monomeric compounds of the formula (2)

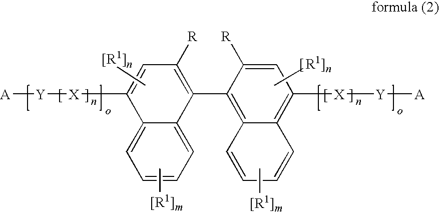

characterized in that the two functional A groups, being the same or different at each instance, copolymerize under conditions of C—C or C—N bond formations by polycondensation and are selected from Cl, Br, I, O-tosylate, O-triflate, O—SO₂R², B(OR²)₂ and Sn(R²)₃, preferably from Br, I and B(OR²)₂, where R² is as defined as described above, and where two or more R² radicals together may also form a ring system; the further symbols and indices are each defined as described in formula (1).

The present invention still further provides bifunctional monomeric compounds of the formula (2) as depicted above, with the proviso that the following compounds are excluded from the invention:

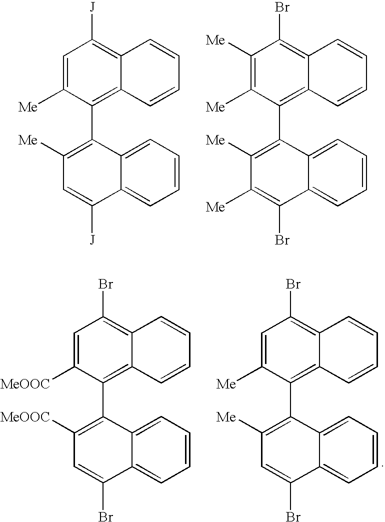

The C—C bond formations are preferably selected from the groups of the SUZUKI coupling, the YAMAMOTO coupling and the STILLE coupling; the C—N bond formation is preferably a coupling according to HARTWIG-BUCHWALD. Particular preference is given to SUZUKI coupling.

For bifunctional monomeric compounds of the formula (2), the same preferences apply as for the structural units of the formula (1).

The monomers of the formula (2) are present in two enantiomeric forms. The invention encompasses both the two enantiomerically pure forms and the racemate, and also mixtures in which one of the two enantiomers is present in enriched form.

It may be preferred to use the inventive polymer not as a pure substance but rather as a mixture (blend) together with further polymeric, oligomeric, dendritic or low molecular weight substances. These may, for example, improve the electronic properties, influence the transfer from the singlet to the triplet state or themselves emit light. For example, a preferred embodiment of the invention is the addition of a compound which can emit light from the triplet state at room temperature, so that the mixture is capable of emitting light from the triplet state with high efficiency. Such compounds have already been described above as further structural elements for the polymer. For this added triplet emitter, the same preferences apply as already described above. Electronically inert substances may also be advisable in order, for example, to influence the morphology of the polymer film formed or the viscosity of polymer solutions. Such mixtures therefore also form part of the subject-matter of the present invention.

The invention further provides solutions and formulations composed of one or more inventive polymers or blends in one or more solvents. The way in which polymer solutions can be prepared is described, for example, in WO 02/072714, in WO 03/019694 and in the literature cited therein.

The inventive polymers may be used in PLEDs. These comprise cathode, anode, emission layer and optionally further layers, for example preferably a hole injection layer and optionally an intermediate layer between the hole injection layer and the emission layer. The way in which PLEDs can be produced is described in WO 04/037887 comprehensively as a general process which is adaptable appropriately for the particular case.

As described above, the inventive polymers are very particularly suitable as electroluminescent materials in PLEDs or displays produced in this way.

In the context of the invention, electroluminescent materials are regarded as being materials which can find use as an active layer in a PLED. Active layer means that the layer is capable of emitting light on application of an electrical field (light-emitting layer) and/or that it improves the injection and/or the transport of the positive and/or negative charges (charge injection or charge transport layer). It may also be an intermediate layer between a hole injection layer and an emission layer. It is preferably an emitting layer.

The invention therefore also provides for the use of an inventive polymer or blend in a PLED, especially as an electroluminescent material.

The invention likewise provides a PLED having one or more active layers, at least one of these active layers comprising one or more inventive polymers or blends. The active layer may, for example, be a light-emitting layer and/or a transport layer and/or a charge injection layer and/or an intermediate layer.

Compared to the polyspirobifluorenes described in WO 03/020790 and polyfluorenes described in WO 02/077060, which are hereby specified as the closest prior art, the inventive polymers have the following surprising advantages:
(1) It has been found that the inventive polymers which contain units of the formula (1) in addition to other basic structure units have distinctly better solubility. This makes it possible to obtain polymers which are soluble in a wider range of solvents, and it is possible to dispense with the use, hitherto necessary in some cases, of chlorobenzene, which is ecologically controversial and cannot be used in industrial processing steps, as a solvent. A particular advantage has been found to be that no long alkyl or alkoxy chains which contribute nothing to the electronic function of the polymer and lower the concentration of the functional units in the polymer are needed on the units of the formula (1).
(2) In addition, it has been found that, surprisingly, again in direct comparison, the inventive polymers in combination with triplet emitters have higher efficiency. This relates in particular also to green triplet emitters. Moreover, triplet emission with greater colour purity is obtained, since only the emission of the triplet emitter is observed in the inventive polymers, but not the residual emission of the polymer backbone, which is still the case in some prior art polymers.
(3) The obtainability and the achievability of colours is equal or better for the inventive polymers in comparison to the prior art. Especially in the case of blue-emitting polymers, an improved colour locus and a more saturated blue emission are observed. The emission bands are also sharper, the reason for which is possibly the reduced conjugation of the polymer.
(4) Since the novel polymer backbone of the formula (1) itself leads to deep blue emission, it is readily possible to introduce certain emitting units which lead to blue emission in the polymer. This makes it possible to separate charge transport and emission properties in the polymer. We believe that this is necessary to obtain stable polymers. To date, this was only possible with difficulty since the polymer backbone itself had always emitted at the same time.

In the present application text and also in the examples which follow below, the aim is the use of the inventive polymers or blends in relation to PLEDs and the corresponding displays. In spite of this restriction of the description, it is possible for those skilled in the art without any inventive activity also to utilize the inventive polymers for further uses in other electronic devices, for example for organic integrated circuits (O-ICs), organic field-effect transistors (O-FETs), organic thin-film transistors (O-TFTs), organic solar cells (O-SCs), organic field-quench devices (O-FQDs), organic light-emitting transistors (O-LETs) or organic laser diodes (O-lasers), to name just a few applications.

The use of the inventive polymers in the corresponding devices, just like these devices themselves, likewise form part of the subject-matter of the present invention.

### EXAMPLES

### Example 1

### Synthesis of Monomers which Lead to Units of the Formula (1) in Polymers

### 1.1 Preparation of 2,2-substituted 4,4′-linkable 1,1′-binaphthalenes

### 1.1.1 Preparation of 2-(pentamethylphenyl)naphthalene

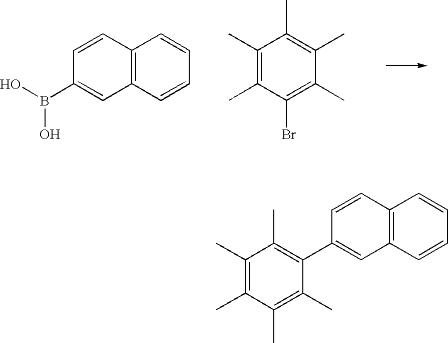

44.3 g (195 mmol) of bromopentamethylbenzene, 45.2 g (263 mmol) of naphthalene-2-boronic acid and 120 g (522 mmol) of potassium phosphate are suspended in 120 ml of toluene, 120 ml of dioxane and 240 ml of water, and saturated with N₂ for 30 min. Subsequently, 2.96 g (9.71 mmol) of o-tolylphosphine and, after stirring for 5 min, 365 mg (1.62 mmol) of Pd(OAc)₂ are added. The reaction mixture is heated under reflux for 1 h. After cooling to RT, the mixture is extended with ethyl acetate, and the organic phase is removed, washed with water, dried over Na₂SO₄ and concentrated by rotary evaporation. For further purification, the residue is recrystallized from EtOH. The yield is 40 g (75%).

### 1.1.2 Preparation of 1-bromo-2-(pentamethylphenyl)naphthalene

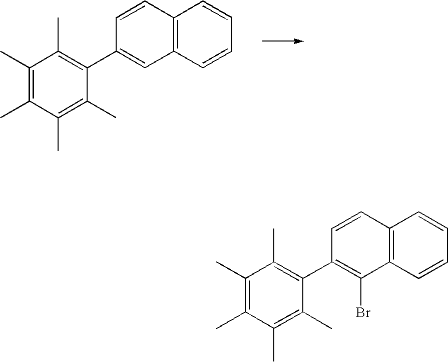

46.1 g (168 mmol) of 2-(pentamethylphenyl)naphthalene are dissolved in 350 ml of CHCl₃ and cooled to 5° C. A solution of 8.6 ml (168 mmol) of bromine in 40 ml of CHCl₃ is added dropwise at this temperature within 1 h. After 1 h, 50 ml of saturated Na₂SO₃ solution are added dropwise, the organic phase is removed, washed twice with water and dried over Na₂SO₄, and the solvent is removed in vacuo. After recrystallization from EtOH/toluene, the bromide is obtained in the form of colourless crystals (52.2 g, 88%).

### 1.1.3 Preparation of 2-pentamethylphenyl-1-(1-naphthyl)naphthalene

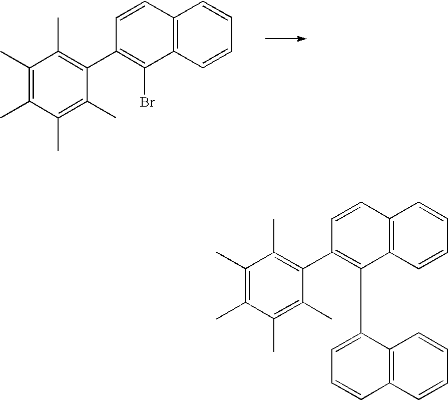

10.2 g (59 mmol) of 1-naphthylboronic acid, 10.4 g (29.4 mmol) of 1-bromo-2-(pentamethylphenyl)naphthalene and 29.9 g (130 mmol) of potassium phosphate are suspended in 35 ml of toluene, 35 ml of dioxane and 70 ml of water, and saturated with N₂ for 30 min. Subsequently, 550 mg (1.8 mmol) of o-tolylphosphine and, after stirring for 5 min, 67 mg (0.3 mmol) of Pd(OAc)₂ are added. The reaction mixture is heated under reflux for 2 h. After cooling to RT, the mixture is extended with ethyl acetate, and the organic phase is removed, washed three times with water, dried over Na₂SO₄ and freed of the solvent in vacuo. The remaining oil is crystallized from i-PrOH. 10 g (82%) yield in the form of a colourless powder.

### 1.1.4 Preparation of 4,4′-dibromo-2-pentamethylphenyl-1-(1-naphthyl)-naphthalene (M1)

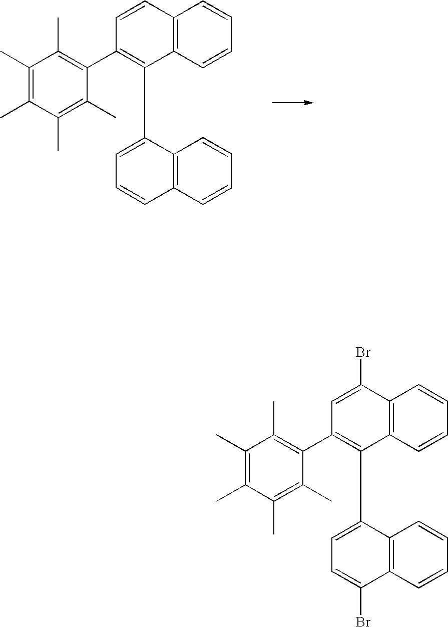

9 g (22 mmol) of 2-pentamethylphenyl-1-(1-naphthyl)naphthalene are dissolved in 100 ml of CHCl₃ and cooled to 5° C. A solution of 2.2 ml (44 mmol) of bromine in 10 ml of CHCl₃ is added dropwise at this temperature within 1 h. After 1 h, 20 ml of saturated Na₂SO₃ solution are added dropwise, the organic phase is removed, washed twice with water and dried over Na₂SO₄, and the solvent is removed in vacuo. After recrystallizing three times from EtOH/toluene, the bromide is obtained in the form of colourless crystals with an HPLC purity of >99.8% (8.3 g, 66%).

### 1.1.6 Preparation of 4,4′-(2-[1,3,2]dioxaborolanyl)-2-pentamethylphenyl-1-(1-naphthyl)naphthalene (M2)

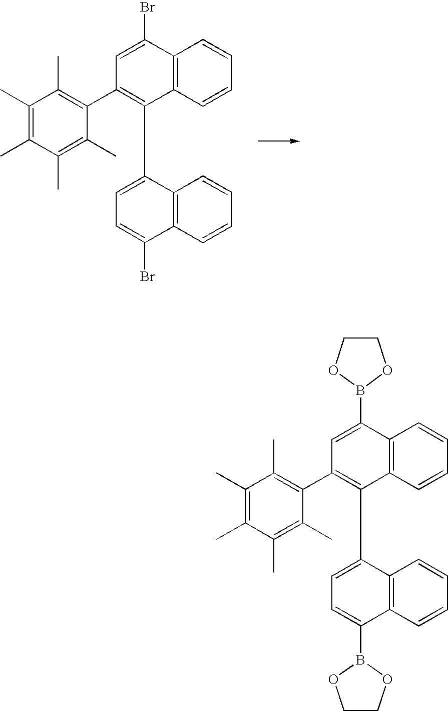

5.8 g of magnesium (235 mmol) are initially charged in a dried apparatus and the Grignard reagent is prepared from 67.7 g (112 mmol) of dibromide (from Example 1.1.4) in 340 ml of THF under reflux. After 3 h, the solution is cooled and added dropwise at −75° C. to the solution of 38 ml (336 mmol) of trimethyl borate in 250 ml of THF. The reaction mixture is brought to RT over a period of 6 h, 100 ml of ethyl acetate, 30 ml of glacial acetic acid and 60 ml of H₂O are added, and the organic phase is removed, washed twice with water and dried over Na₂SO₄. The solid remaining after the removal of the solvent is suspended in 200 ml of toluene, 12.5 ml of anhydrous ethylene glycol are added and the suspension is heated to vigorous boiling on a water separator for 4 h. After again removing the solvent, the remaining solid is recrystallized four times from acetonitrile. The diester is obtained as colourless crystals having a purity of >99.9%; the yield is 36 g (58%).

### 1.1.6 Preparation of 4,4′-dibromo-2,2′-dimethyl-1,1′-binaphthyl (M3)

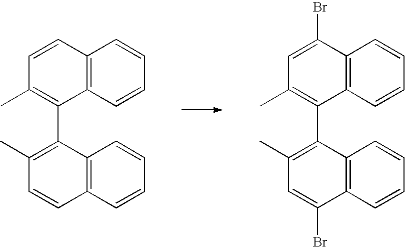

59.2 g (200 mmol) of 2,2′-dimethylbinaphthyl (prepared analogously to N. Maigrot, J. P. Mazaleyrat, Synthesis 1958, 317) are dissolved in 1000 ml of CHCl₃ and cooled to 5° C. A solution of 20 ml (400 mmol) of bromine in 90 ml of CHCl₃ is added dropwise at this temperature within 1 h. After 1 h, 180 ml of saturated Na₂SO₃ solution are added dropwise, the organic phase is removed, washed twice with water and dried over Na₂SO₄, and the solvent is removed in vacuo. After repeated recrystallization from i-PrOH, the bromide is obtained in the form of colourless crystals having an HPLC purity of >99.7% (50.9 g, 56%).

### 1.1.7 Preparation of 4,4′-(2-[1,3,2]dioxaborolanyl)-2,2′-dimethyl-1,1′-binaphthyl (M4)

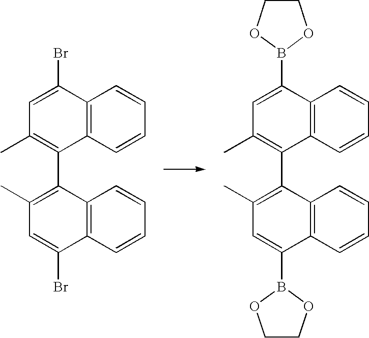

5.8 g of magnesium (235 mmol) are initially charged in a dried apparatus and the Grignard reagent is prepared from 50.8 g (112 mmol) of dibromide (from Example 1.1.6) in 340 ml of THF under reflux. After 3 h, the solution is cooled and added dropwise at −75° C. to the solution of 38 ml (336 mmol) of trimethyl borate in 250 ml of THF. The reaction mixture is brought to RT over a period of 6 h, 100 ml of ethyl acetate, 30 ml of glacial acetic acid and 60 ml of H₂O are added, and the organic phase is removed, washed twice with water and dried over Na₂SO₄. The solid remaining after the removal of the solvent is suspended in 200 ml of toluene, 12.5 ml of anhydrous ethylene glycol are added and the suspension is heated to vigorous boiling on a water separator for 4 h. After again removing the solvent, the remaining solid is recrystallized four times from acetonitrile. The diester is obtained as colourless crystals having a purity of >99.8%; the yield is 67.5 g (62%).

### 1.1.8 Preparation of 2,2′-dioctyl-1,1′-binaphthyl

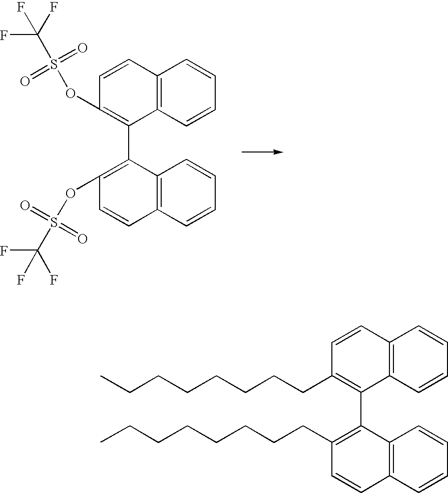

The Grignard reagent prepared from 70 g (364 mmol) of octyl bromide and 8.85 g (364 mmol) of magnesium in 350 ml THF is added dropwise at RT to 50 g (91 mmol) of 2,2′-bis(trifluoromethanesulphonyl)-1,1′-binaphthyl, 4.88 g (9.1 mmol) of NiCl₂dppp are added and the mixture is heated under reflux for 24 h. 20 ml of acetic acid are then added dropwise with ice cooling, the mixture is worked up extractively with heptane and water, and the organic phase is dried over Na₂SO₄. After column filtration on silica gel with heptane, the oily residue remaining after removal of the solvent is purified by means of short-path distillation. 38.3 g (88%) of a yellowish, highly viscous oil are obtained.

### 1.1.9 Preparation of 4,4′-dibromo-2,2′-dioctyl-1,1′-binaphthyl (M5)

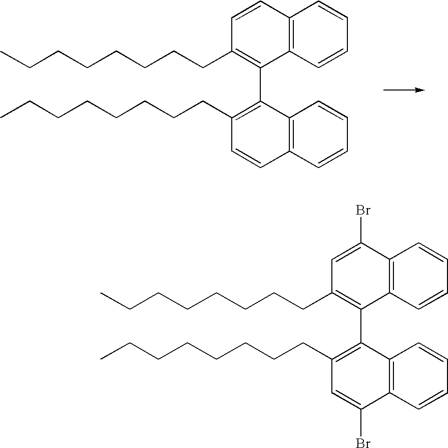

38 g (79 mmol) of 2,2′-dioctyl-1,1′-binaphthyl are dissolved in 400 ml of CHCl₃ and cooled to 5° C. A solution of 8 ml (160 mmol) of bromine in 35 ml of CHCl₃ is added dropwise at this temperature within 1 h. After 1 h, 75 ml of saturated Na₂SO₃ solution are added dropwise, the organic phase is removed, washed twice with water and dried over Na₂SO₄, and the solvent is removed in vacuo. The remaining residue is purified by means of preparative HPLC. The dibromide is then obtained in the form of a colourless, viscous oil with a purity of >99.8% and a yield of 38 g (76%).

### 1.1.10 Preparation of 4,4′-(2-[1,3,2]dioxaborolanyl)-2,2′-dioctyl-1,1′-binaphthyl (M6)

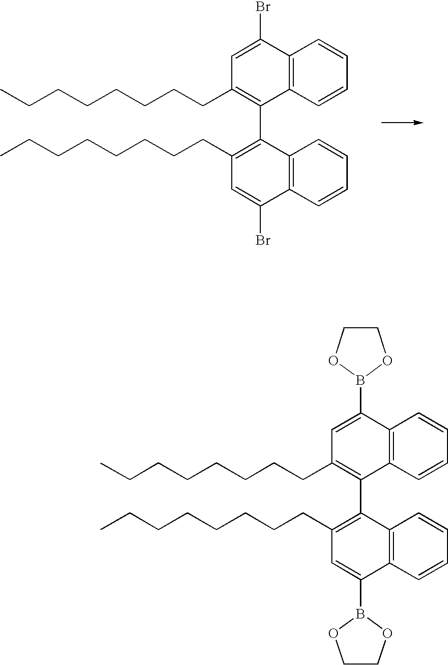

2.9 g of magnesium (118 mmol) are initially charged in a dried apparatus and the Grignard reagent is prepared from 35.7 g (56 mmol) of dibromide (from Example 1.1.9) in 170 ml of THF under reflux. After 3 h, the solution is cooled and added dropwise at −75° C. to the solution of 19 ml (168 mmol) of trimethyl borate in 125 ml of THF. The reaction mixture is brought to RT over a period of 6 h, 50 ml of ethyl acetate, 15 ml of glacial acetic acid and 30 ml of H₂O are added, and the organic phase is removed, washed twice with water and dried over Na₂SO₄. The solid remaining after the removal of the solvent is suspended in 100 ml of toluene, 6.5 ml of anhydrous ethylene glycol are added and the solution is heated to vigorous boiling on the water separator for 4 h. After again removing the solvent, the remaining solid is extracted once from boiling acetonitrile and recrystallized three times from pentane. The diester is obtained as colourless crystals having a purity of >99.6%; the yield is 25.6 g (74%).

### Example 2

### Synthesis of Further Monomers

The synthesis of monomers M7 to M10 is described in WO 03/020790 and the literature cited therein.

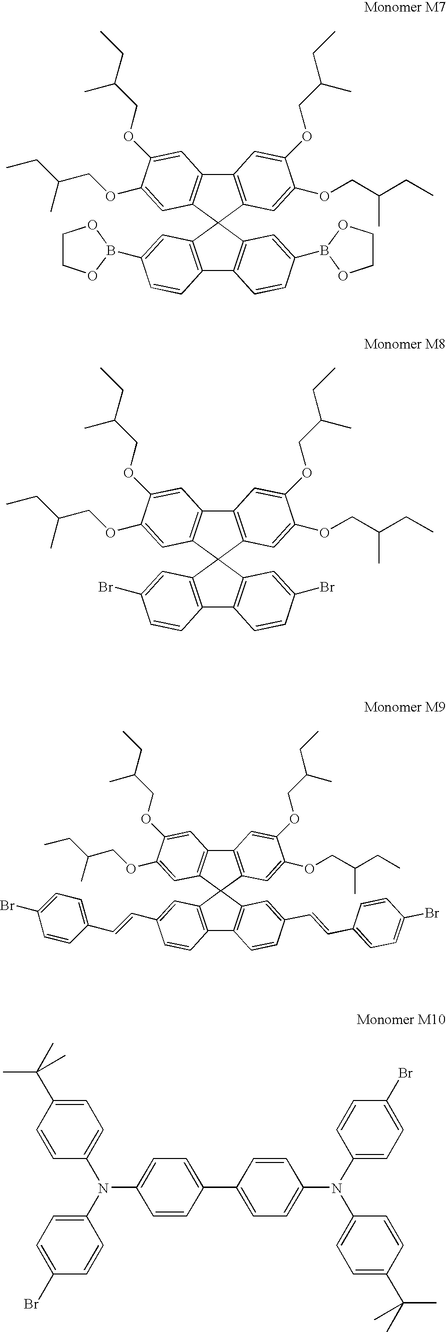

### Example 3

### Synthesis of the Polymers

The polymers were synthesized by SUZUKI coupling according to WO 03/048225. The composition of the synthesized polymers P1 to P5 is compiled in Table 1. Also synthesized were the comparative polymers C1 and C2 which, instead of the monomer M1-6 which leads to units of the formula (1) in the polymer, contain the monomer M8 or/and M7. The composition of these comparative polymers is likewise listed in Table 1.

### Example 4

### Production of the PLEDs

The polymers were investigated for use in PLEDs. The PLEDs were each two-layer systems, i.e. substrate//ITO//PEDOT//polymer//cathode. PEDOT is a polythiophene derivative (Baytron P from H. C. Starck, Goslar). The cathode used in all cases was Ba/Ag (Aldrich). The way in which PLEDs can be prepared is described in detail in WO 04/037887 and the literature cited therein.

### Example 5 to 11

### Device Examples

The results which were obtained on use of the polymers P1 to P5 in PLEDs are compiled in Table 1. Likewise listed are the electroluminescence results which were obtained using the comparative polymers C1 to C2.

As can be seen from the results, the emission colour of the inventive polymers is shifted to deeper blue, and the lifetimes are, especially based on the emission colour, distinctly improved. This shows that the inventive polymers are better suited to use in displays than prior art polymers.

TABLE 1
Device results with inventive polymers and comparative polymers

| | | Monomer for | | | | | |
|---|---|---|---|---|---|---|---|
| | | units of the | | Max. Eff./ | U @ | | |
| Example | Polymer | formula (1) | Further monomers | cd/A | 100 cd/m²/V | CIE x/y^{a} | Lifetime^{b}/h |
| 5 | P1 | 10% M3 | 50% M7, 30% M8, | 2.70 | 4.6 | 0.15/0.15 | 100 |
| | | | 10% M10 | | | | |
| 6 | P2 | 10% M3 | 50% M7, 20% M8, | 4.50 | 4.0 | 0.16/0.27 | 610 |
| | | | 10% M10, 10% M9 | | | | |
| 7 | P3 | 50% M6 | 30% M8, 10% M10, | 2.64 | 5.1 | 0.15/0.18 | 400 |
| | | | 10% M9 | | | | |
| 8 | P4 | 50% M2, | 10% M10 | 1.91 | 5.5 | 0.14/0.12 | 78 |
| | | 40% M5 | | | | | |
| 9 | P5 | 50% M2, | 10% M10, 10% M9 | 2.32 | 4.8 | 0.14/0.16 | 354 |
| | | 30% M5 | | | | | |
| 10 | C1 | — | 50% M7, 40% M8, | 2.86 | 4.4 | 0.16/0.18 | 80 |
| (Comparative) | | | 10% M10 | | | | |
| 11 | C2 | — | 50% M7, 30% M8, | 4.66 | 3.9 | 0.17/0.30 | 530 |
| (Comparative) | | | 10% M10, 10% M9 | | | | |

^{a}CIE coordinates: colour coordinates of the Commision Internationale de I'Eclairage 1931.
^{b}Lifetime: time until decline of the brightness to 50% of the starting brightness, starting brightness 400 cd/m².

1-24. (canceled)

25. A polymer comprising at least 1 mol % of a first repeat unit of the formula (1) 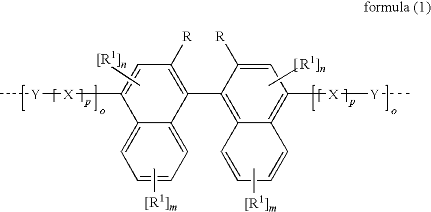
wherein
R identically or differently is H; a straight alkyl chain having up to 40 carbon atoms; or a branched or cyclic alkyl chain having 3 to 40 carbon atoms; wherein said straight, branched, or cyclic alkyl chain is optionally substituted by R¹; and wherein one or more nonadjacent carbon atoms of said straight, branched, or cyclic alkyl chain is optionally replaced by —N—R¹—, —O—, —S—, —O—CO—O—, —CO—O—, —CR¹═CR¹—, or —C≡C—, with the proviso that the heteroatoms are not bonded directly to the naphthyl unit; and wherein one or more hydrogen atoms are optionally replaced by F; Cl; Br; I; CN; or an aromatic or heteroaromatic ring system having from 5 to 40 aromatic ring atoms, optionally substituted by one or more R¹ radicals; and wherein two R optionally define a ring system; with the proviso that at least one of the two R is not H;
X identically or differently is —CR¹═CR¹—, —C≡C—, or N—Ar;
Y identically or differently is a bivalent aromatic or heteroaromatic ring system having 5 to 40 aromatic ring atoms, optionally substituted by one or more R¹ radicals;
R¹ identically or differently is H; F; Cl; Br; I; CN; N(R²)₂; Si(R²)₃; B(R²)₂; a straight alkyl or alkoxy chain having up to 40 carbon atoms, or a branched or cyclic alkyl or alkoxy chain having 3 to 40 carbon atoms, wherein one or more nonadjacent carbon atoms of said straight alkyl or alkoxy chain or a branched or cyclic alkyl or alkoxy chain is optionally replaced by —N—R²—, —O—, —S—, —O—CO—O—, —CO—O—, —CR¹═CR¹—, or —C≡C—, and wherein one or more hydrogen atoms is optionally replaced by F; Cl; Br; I; CN; or an aryl, aryloxy or heteroaryl group having 5 to 40 aromatic ring atoms, optionally substituted by one or more nonaromatic R¹ radicals; and wherein two or more of R¹ optionally define a ring system;
R² identically or differently is H or an aliphatic or aromatic hydrocarbon radical having up to 20 carbon atoms;
Ar identically or differently is a monovalent aromatic or heteroaromatic ring system having 5 to 40 aromatic ring atoms, optionally substituted by R¹;
n identically or differently is 0 or 1;
m identically or differently is 0, 1, 2, 3, or 4;
o identically or differently is 0, 1, or 2;
p identically or differently is 0 or 1; and
the dashed bond is the linkage in the polymer;
and
at least 1 mol % of a second repeat unit, which is either the same as a repeat unit of the formula (1) or is different.

26. The polymer of claim 25, wherein said polymer is a conjugated or part-conjugated polymer.

27. The polymer of claim 25, wherein
R identically or differently is H; a straight alkyl chain having up to 10 carbon atoms; or a branched or cyclic alkyl chain having 3 to 10 carbon atoms, wherein one or more nonadjacent carbon atoms of said straight alkyl, branched, or cyclic alkyl chain is optionally replaced by —CH═CH— or —C≡C—, and wherein one or more hydrogen atoms is optionally replaced by F; CN; or an aromatic or heteroaromatic ring system having 5 to 20 aromatic ring atoms, optionally substituted by one or more nonaromatic R¹ radicals; and wherein two of R optionally define ring system, with the proviso that at least one R is not H;
Y identically or differently is a bivalent aryl or heteroaryl group having 5 to 20 aromatic ring atoms, optionally substituted by one or more R¹ radicals;
Ar identically or differently is a monovalent aryl or heteroaryl group having 5 to 20 aromatic ring atoms, optionally substituted by R¹;
R¹ identically or differently is H; F; N(R²)₂; a straight alkyl chain having up to 10 carbon atoms; or a branched alkyl chain having 3 to 10 carbon atoms; wherein one or more nonadjacent carbon atoms of said straight or branched alkyl chain is optionally replaced by —CR¹═CR¹— or —C≡C—, and wherein one or more hydrogen atoms is optionally replaced by F, or an aryl or heteroaryl group having 5 to 20 aromatic ring atoms, optionally substituted by one or more nonaromatic R¹ radicals; and wherein two or more of R¹ optionally define a ring system;
n is 0;
m identically or differently is 0, 1, or 2; and
o identically or differently is 0 or 1.

28. The polymer of claim 25, wherein each R is identical.

29. The polymer of claim 25, wherein one R is H and the other R is an aromatic or heteroaromatic ring system having 5 to 15 aromatic ring atoms.

30. The polymer of claim 25, wherein said polymer comprises further structural elements selected from the group consisting of fluorene derivatives, spirobifluorene derivatives, dihydrophenanthrene derivatives, cis-indenofluorene derivatives, trans-indenofluorene derivatives, 1,4-phenylene derivatives, 4,4′-biphenylylene derivatives, 4,4″-terphenylylene derivatives, 2,7-phenanthrene derivatives, 3,6-phenanthrene derivatives, dihydropyrene derivatives, and tetrahydropyrene derivatives, and wherein said further structural elements are optionally substituted with R¹.

31. The polymer of claim 25, wherein said polymer comprises further structural elements selected from the group consisting of R¹-substituted fused aromatic structures having 6 to 40 carbon atoms, unsubstituted fused aromatic structures having 6 to 40 carbon atoms, tolane derivatives, stilbene derivatives, and bisstyrylarylene derivatives.

32. The polymer of claim 25, wherein said polymer comprises further structural elements selected from the group consisting of R¹-substituted triarylamines, unsubstituted triarylamines, benzidines, N,N,N′,N′-tetraaryl-para-phenylenediamines, triarylphosphines, phenothiazines, phenoxazines, dihydrophenazines, thianthrenes, dibenzo-p-dioxins, phenoxathiines, carbazoles, azulenes, thiophenes, pyrroles, furans, additional O-containing heterocycles with a high-lying HOMO, additional S-containing heterocycles with a high-lying HOMO, and additional N-containing heterocycles with a high-lying HOMO.

33. The polymer of claim 25, wherein said polymer comprises further structural elements selected from the group consisting of R¹-substituted pyridines, unsubstituted pyridines, pyrimidines, pyridazines, pyrazines, triazines, oxadiazoles, quinolines, quinoxalines, phenazines, triarylboranes, additional O-containing heterocycles with a low-lying LUMO, additional S-containing heterocycles with a low-lying LUMO, and additional N-containing heterocycles with a low-lying LUMO.

34. The polymer of claim 25, further comprising structural elements which emit light from the triplet state with high efficiency at room temperature and exhibit electrophosphorescence instead of electrofluorescence and which contain at least one heavy atom having an atomic number of more than 36.

35. The polymer of claim 34, further comprising structural elements selected from the group consisting of carbazoles, bridged carbazole dimers, ketones, phosphine oxides, sulphoxides, sulphones, and silanes.

36. The polymer of claim 25, wherein the proportion of units of the formula (1) present in said polymer is at least 5 mol %.

37. The polymer of claim 25, wherein said unit of the formula (1) forms the backbone of said polymer or is combined with the backbone of said polymer, and wherein p is 0.

38. The polymer of claim 25, wherein said unit of the formula (1) is a hole-transporting unit, and wherein
p identically or differently is 0 or 1, with the proviso that at least one p is 1;
o identically or differently is 0, 1, or 2, with the proviso that o is not 0 when the corresponding p is 1; and
X is N—Ar.

39. The polymer of claim 25, wherein said unit of the formula (1) is an emitter, and wherein
p identically or differently is 0 or 1, with the proviso that at least one p is 1;
o identically or differently is 0, 1, or 2, with the proviso that o is not 0 when the corresponding p is 1; and
X identically or differently is —CR¹═CR¹—, —C≡C—, or N—Ar, with the proviso that at least one X is —CR¹═CR¹— or —C≡C—.

40. A bifunctional monomeric compound of the formula (2) 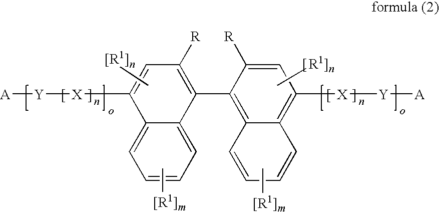
wherein
A identically or differently are Cl, Br, I, O-tosylate, O-triflate, O—SO₂R², B(OR²)₂, or Sn(R²)₃ and wherein both A groups copolymerize under conditions of C—C or C—N bond formation;
R identically or differently is H; a straight alkyl chain having up to 40 carbon atoms;
or a branched or cyclic alkyl chain having 3 to 40 carbon atoms; wherein said straight, branched, or cyclic alkyl chain is optionally substituted by R¹; and wherein one or more nonadjacent carbon atoms of said straight, branched, or cyclic alkyl chain is optionally replaced by —N—R¹—, —O—, —S—, —O—CO—O—, —CO—O—, —CR¹═CR¹—, or —C≡C—, with the proviso that the heteroatoms are not bonded directly to the naphthyl unit; and wherein one or more hydrogen atoms are optionally replaced by F; Cl; Br; I; CN; or an aromatic or heteroaromatic ring system having from 5 to 40 aromatic ring atoms, optionally substituted by one or more R¹ radicals; and wherein two R optionally define a ring system; with the proviso that at least one of the two R is not H;
X identically or differently is —CR¹═CR¹—, —C≡C—, or N—Ar;
Y identically or differently is a bivalent aromatic or heteroaromatic ring system having 5 to 40 aromatic ring atoms, optionally substituted by one or more R¹ radicals;
R¹ identically or differently is H; F; Cl; Br; I; CN; N(R²)₂; Si(R²)₃; B(R²)₂; a straight alkyl or alkoxy chain having up to 40 carbon atoms, or a branched or cyclic alkyl or alkoxy chain having 3 to 40 carbon atoms, wherein one or more nonadjacent carbon atoms of said straight alkyl or alkoxy chain or a branched or cyclic alkyl or alkoxy chain is optionally replaced by —N—R²—, —O—, —S—, —O—CO—O—, —CO—O—, —CR¹═CR¹—, or —C≡C—, and wherein one or more hydrogen atoms is optionally replaced by F; Cl; Br; I; CN; or an aryl, aryloxy or heteroaryl group having 5 to 40 aromatic ring atoms, optionally substituted by one or more nonaromatic R¹ radicals; and wherein two or more of R¹ optionally define a ring system;
R² identically or differently is H or an aliphatic or aromatic hydrocarbon radical having up to 20 carbon atoms and wherein two or more R² optionally define a ring system;
Ar identically or differently is a monovalent aromatic or heteroaromatic ring system having 5 to 40 aromatic ring atoms, optionally substituted by R¹;
n identically or differently is 0 or 1;
m identically or differently is 0, 1, 2, 3, or 4;
o identically or differently is 0, 1, or 2; and
p identically or differently is 0 or 1.

41. The bifunctional monomeric compound of claim 40, with the proviso that said bifunctional monomeric compound of the formula (2) is not any following compounds:

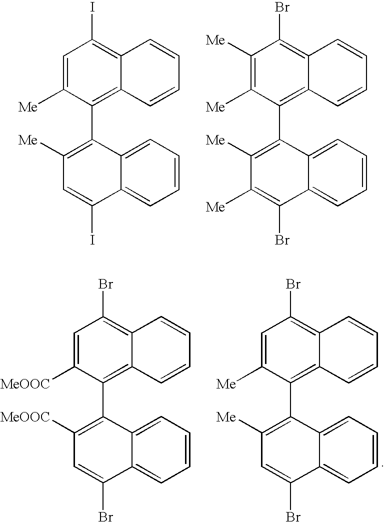

42. A mixture of one or more polymers according to claim 25 with one or more additional polymeric, oligomeric, dendritic, or low molecular weight substance.

43. The mixture of claim 42, further comprising a compound which can emit light from the triplet state at room temperature.

44. A solution or formulation comprising one or more polymers according to claim 25 and one or more solvents.

45. An organic electronic device comprising one or more active layers, wherein at least one of said one or more active layers comprises one or more polymers according to claim 25.

46. The organic electronic device of claim 45, wherein said organic electronic device is selected from the group consisting of polymeric light-emitting diodes, organic integrated circuits, organic field-effect transistors, organic thin-film transistors, organic solar cells, organic field-quench devices, organic light-emitting transistors, and organic laser diodes.
